(19) United States
(12) Patent Application Publication
(10) Pub. No.: US 2024/0389915 A1
(43) Pub. Date: Nov. 28, 2024

(54) SCREENING FOR AUTISM SPECTRUM DISORDER

(71) Applicant: Welch Allyn, Inc., Skaneateles Falls, NY (US)

(72) Inventor: Vivian Loomis Hunter, Auburn, NY (US)

(21) Appl. No.: 18/656,842

(22) Filed: May 7, 2024

Related U.S. Application Data
(60) Provisional application No. 63/503,537, filed on May 22, 2023.

(51) Int. Cl. A61B 5/00; A61B 3/00; A61B 3/103; A61B 3/11; A61B 3/113; A61B 3/14

(52) U.S. Cl. CPC A61B 5/163; A61B 3/0091; A61B 3/103; A61B 3/112; A61B 3/113; A61B 3/145; A61B 5/168; A61B 3/0041; A61B 2503/06

(57) ABSTRACT

A device for screening for autism spectrum disorder includes a housing configured for portable use, one or more cameras mounted to the housing, and one or more light sources mounted to the housing. The device captures a video of one or more eyes using the one or more cameras, emits a light stimulus using the one or more light sources, and synchronizes the video to the light stimulus. The device analyzes the video for measuring a pupillary response to the light stimulus. The device compares the pupillary response to one or more metrics. The device generates an output based on comparing the pupillary response to the one or more metrics, the output including a recommendation related to autism spectrum disorder.

### BACKGROUND

The autism spectrum disorder is a range of neurodevelopmental conditions generally characterized by difficulties in social interactions and communication, repetitive behaviors, intense interests, and unusual responses to sensory stimuli. The term “spectrum” in autism spectrum disorder refers to the wide range of symptoms and severity.

Autism spectrum disorder begins in early childhood. For example, children can show symptoms of autism within the first year of birth. A small number of children appear to develop normally in the first year, and then go through a period of regression between 18 and 24 months of age when they develop autism symptoms. While there is no cure for autism spectrum disorder, early intervention can impact long-term outcomes such as between being verbal or nonverbal.

### SUMMARY

In general terms, the present disclosure relates to screening for autism spectrum disorder. In one possible configuration, a portable device generates an output by comparing a pupillary response to one or more metrics, the output including a recommendation related to autism spectrum disorder. Various aspects are described in this disclosure, which include, but are not limited to, the following aspects.

One aspect relates to a device for screening for autism spectrum disorder, the device comprising: a housing configured for portable use; one or more cameras mounted to the housing; one or more light sources mounted to the housing; one or more processing devices housed inside the housing; and computer-readable media storing software instructions that, when executed by the one or more processing devices, cause the one or more processing devices to: capture a video of one or more eyes using the one or more cameras; emit a light stimulus using the one or more light sources; synchronize the video to the light stimulus; analyze the video for measuring a pupillary response to the light stimulus; compare the pupillary response to one or more metrics; and generate an output based on comparing the pupillary response to the one or more metrics, the output including a recommendation related to autism spectrum disorder.

Another aspect relates to a method of screening for autism spectrum disorder, the method comprising: capturing a video of one or more eyes; emitting a light stimulus; synchronizing the video to the light stimulus; analyzing the video for measuring a pupillary response to the light stimulus; comparing the pupillary response to one or more metrics; and generating an output based on comparing the pupillary response to the one or more metrics, the output including a recommendation related to autism spectrum disorder.

Another aspect relates to a non-transitory computer-readable media storing data instructions, which when executed by one or more processing devices, cause the one or more processing devices to: capture a video of one or more eyes; emit a light stimulus; synchronize the video to the light stimulus; analyze the video for measuring a pupillary response to the light stimulus; compare the pupillary response to one or more metrics; and generate an output based on comparing the pupillary response to the one or more metrics, the output including a recommendation related to autism spectrum disorder.

A variety of additional aspects will be set forth in the description that follows. The aspects can relate to individual features and to combinations of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of an environment that includes a screening device for administering screening tests including at least a screening test for autism spectrum disorder.

FIG. 2 illustrates an example of the screening device of FIG. 1.

FIG. 3 illustrates an expanded view of an array of light-emitting diodes (LEDs) on the screening device of FIG. 2.

FIG. 4 illustrates an example of the screening device of FIG. 1 being used by an operator to administer screening tests on a patient.

FIG. 5 illustrates an example of components of the screening device of FIG. 1 being used for an autism spectrum disorder screening test on the patient.

FIG. 6 schematically illustrates an example of a method of screening for autism spectrum disorder than can be performed in the screening device of FIGS. 1-5.

### DETAILED DESCRIPTION

The present disclosure is directed to screening for autism spectrum disorder. As will be described in more detail, the screening for autism spectrum disorder is performed on a device that can also be used to perform screening for eye vision, eye diseases, and other abnormalities.

Typically, autism spectrum disorder is diagnosed based on parent or clinician questionnaires that include questions about the child's behavior, and the answers to such questions are often subjective. Also, such questionnaires are typically performed around four years of age. The screening for autism spectrum disorder described herein can be performed earlier than typical autism screening tests to enable earlier intervention. Also, the screening for autism spectrum disorder described herein removes subjectivity from the diagnosis.

The device on which the screening for autism spectrum disorder is performed can be handheld and portable and can screen for autism spectrum disorder a distance away from a patient such as a child for patient comfort. The device can output the results of an autism screening test and other tests for screening for eye vision, diseases, and abnormalities to an operator of the device, such as a clinician or a physician's assistant. The device can incorporate aspects of the vision screening device described in U.S. patent application Ser. No. 18/099,062, filed Jan. 19, 2023, the entire disclosure of which is incorporated herein by reference.

Based at least in part on an analysis of the patient's response to a stimulus generated by the device, the device can generate an output including at least one of a recommendation or a diagnosis associated with the patient. Such output can indicate a likelihood of autism spectrum disorder detection, indicate that the patient requires additional screening, or indicate that the patient's response was normal (e.g., did not indicate a likelihood of autism spectrum disorder).

The device can measure a response in the left and right eyes of the patient to the stimulus and can thereafter compare the measured response to responses collected from other patients having the same age as the patient to generate the output. In some examples, the response in the left and right eyes of the patient is compared to one or more thresholds or value ranges, and the output generated by the device is based on the response exceeding the one or more thresholds or being outside of the value ranges.

In view of the foregoing, the device can provide an automated recommendation or diagnosis for autism spectrum disorder based on the analysis of the response in the left and right eyes of the patient to a stimulus. The device can also generate visualizations of the captured response for display to assist the clinician or the operator in determining a manual diagnosis.

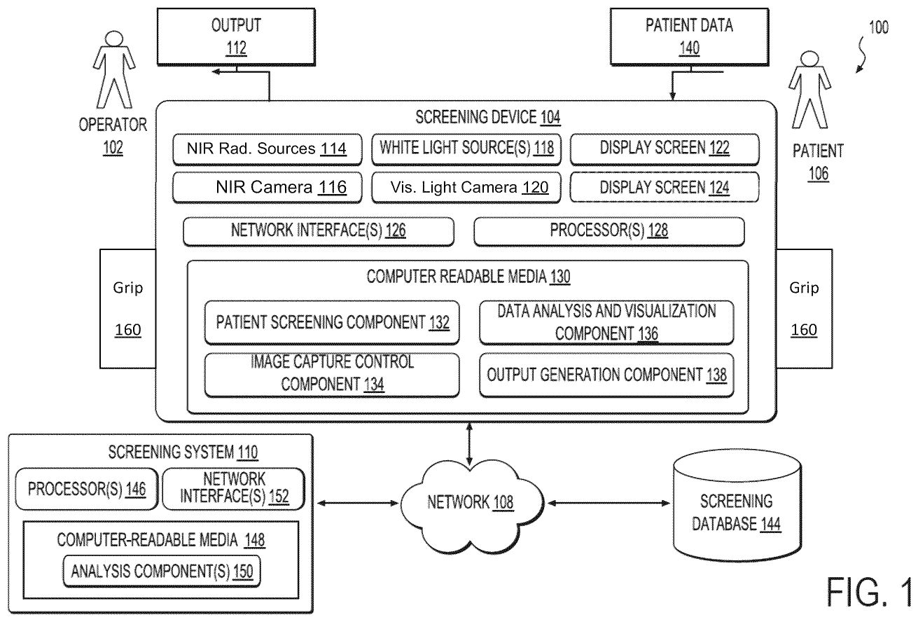

As will be described with respect to at least FIG. 1, an example device can include components associated with screening for eye vision, diseases, and abnormalities, which can also be used for screening for autism spectrum disorder. For example, the device can include components for controlling emission of near-infrared and visible light radiation, and the corresponding capture of the reflected radiation from the eyes during the screening.

In some examples, near-infrared images are captured before initiating the capture of visible light images, and the screening can be completed without the need for dilation of the eyes. The device can further include components for reporting the outputs indicating the diagnoses, recommendations, and/or abnormalities detected during the screening.

Additional details pertaining to the above-mentioned devices and techniques are described below. It is to be appreciated that while these figures describe devices and systems that can utilize the claimed methods, operations, and/or techniques described herein, such methods, operations, and/or techniques can apply to other devices, systems, and the like.

FIG. 1 illustrates an example of an environment 100 for administering screening tests including at least a screening test for autism spectrum disorder. The environment 100 can also be used for administrating vision screening tests, and in particular, screening tests for detection of eye diseases and/or abnormalities, according to some implementations.

As illustrated in FIG. 1, an operator 102 can administer an autism spectrum disorder screening test on a patient 106 via a screening device 104. The screening device 104 can also perform one or more vision screening tests, including screening for eye diseases and/or abnormalities. For example, the screening device 104 can be configured to perform vision screening tests such as visual acuity tests, refractive error tests, eye accommodation tests, dynamic eye tracking tests, color vision screening tests, and/or any other vision screening tests, can be further configured to evaluate and/or diagnose the eye health of the patient 106.

In some examples, the screening device 104 is a portable device such that the screening device 104 can perform the autism and/or vision screening tests at any location, from conventional screening environments, such as schools and medical clinics, to physician's offices, hospitals, eye care facilities, and/or other remote and/or mobile locations. It is also envisioned that the screening device 104 can be used for administering vision screening tests to all age groups, including newborns and young children, and adult patients. In some examples, the screening device 104 includes one or more hand grips 160 for the operator 102 to hold the screening device 104 with stability during the screening tests.

As described herein, the screening device 104 can be configured to perform one or more vision screening tests on the patient 106. In some examples, one or more vision screening tests can include illuminating the eyes of the patient 106 with infrared or near-infrared (NIR) radiation and capturing reflected radiation from the eyes of the patient 106. For example, U.S. Pat. No. 9,237,846, the entire disclosure of which is incorporated herein by reference, describes systems and methods for determining refractive error based on photorefraction using pupil images captured under different illumination patterns generated by near-infrared (NIR) radiation sources. In further examples, vision screening tests, such as the red reflex test, can include illuminating the eyes of the patient 106 with visible light, and capturing color images of the eyes under visible light illumination.

The screening device 104 captures data such as infrared images, color images, and/or video data of the eyes. The data can be used to detect anatomical structures of the eye such as pupils, retinas, and/or lenses. The data can also be used to determine differences between the left and right eyes, compare the captured images with standard images, and generate visualizations to assist the operator 102 in diagnosing one or more diseases and abnormalities.

The screening device 104 can transmit the data, via a network 108, to a screening system 110 for analysis to determine an output 112 based on the data captured from the patient 106. The output 112 can be related to eye vision, diseases, and/or abnormalities. Additionally, or alternatively, the output 112 can be related to autism spectrum disorder based on comparing a pupillary response to one or more metrics. In some examples, the disclosed methods and techniques can be performed in whole or in part on the screening system 110 (e.g., with or without the screening device 104).

Alternatively, or in addition, the screening device 104 can perform some or all of the analysis locally to determine the output 112. For example, the disclosed methods and techniques can be performed in whole or in part on the screening device 104 (e.g., with or without the screening system 110). In some instances, the screening device 104 can be configured to perform any of the screening tests described herein without being connected to, or otherwise in communication with, the screening system 110 via the network 108. In other examples, the screening system 110 can be configured to perform any of the screening tests described herein without being connected to, or otherwise in communication with, the screening device 104.

The screening device 104 includes near-infrared (NIR) radiation sources 114 configured to perform functions associated with administering one or more vision screening tests. The NIR radiation sources 114 can include one or more individual radiation emitters, such as light-emitting diodes (LEDs). In some examples, the NIR radiation sources 114 include NIR LEDs arranged in a pattern to form an LED array for measuring the refractive error of the eyes of the patient 106 using photorefraction methods. The NIR radiation sources 114 can also be used for measuring the gaze angle or gaze direction of the eyes of the patient 106.

The screening device 104 can include a near-infrared (NIR) camera 116. As an illustrative example, the NIR camera 116 can capture near-infrared radiation reflected from the eyes of the patient during the screening tests. The screening device 104 can include additional types of sensors such as an ambient light sensor to measure an amount of ambient light during a screening test in a room or location where the screening device is positioned.

As an illustrative example, the screening device 104 can emit, via the NIR radiation sources 114, one or more beams of near-infrared radiation, and can be configured to direct such beams at the eyes of the patient 106. The screening device 104 captures, via the NIR camera 116, near-infrared radiation reflected from pupils of the eyes. Additionally, the NIR camera 116 can capture reflected NIR radiation while the eyes of the patient 106 are illuminated by a visible light source such as visible light sources 118 and/or a second display screen 124, which are described in more detail below. The NIR radiation can be captured intermittently, during specific periods of the screening tests, or during the entire duration of the screening tests.

The NIR radiation captured by the NIR camera 116 can be used to measure refractive error and/or gaze angles of the eyes of the patient 106. Additionally, the NIR radiation captured by the NIR camera 116 can include images and/or video of the pupils of the eyes of the patient 106 for measuring a pupillary response to a light stimulus, which as will be described further below, can be used to screen for autism spectrum disorder.

The screening device 104 includes one or more visible light sources 118 and a visible light camera 120 configured to capture color images and/or video of the eyes under illumination by the visible light sources 118. The visible light sources 118 can comprise light-emitting diodes (LEDs) such as an array of LEDs configured to produce white light e.g., a blue LED with a phosphor coating to convert blue light to white light, or a combination of red, blue, and green LEDs configured to produce white light by varying intensities of individual red, blue, and green LED activation. Individual LEDs of the array of LEDs can be arranged in a pattern configured to be individually operable to provide illumination from different angles during the screening tests. The visible light sources 118 can also be configured to produce white light of different intensity levels. The visible light sources 118 can also include color LEDs for generating color stimuli for display to the patient 106 during a color vision screening test. Also, the visible light sources 118 can be used to generate stimuli for screening for autism spectrum disorder.

The visible light camera 120 can be used by the screening device 104 to record a pupillary response of the patient 106 that results from a stimulus produced by the visible light sources 118 and/or a second display screen 124 (described in more detail below). In some examples, in addition to, or as an alternative to the visible light camera 120, the NIR camera 116 (e.g., infrared camera) can be used to record the pupillary response by the patient 106 that results from a stimulus produced by the visible light sources 118 and/or the second display screen 124.

The visible light camera 120 captures visible light reflected from the eyes of the patient 106 to produce digital color images and/or video. The visible light camera 120 can include a high-resolution, auto-focus digital camera with custom optics for imaging eyes in clinical applications. The color images and/or video captured by the visible light camera 120 can be stored in various formats such as JPEG, BITMAP, TIFF, and the like for images and MP4, MOV, WMV, AVI, and the like for video. In some examples, pixel values in the color images and/or video can be in a RGB (red, green, blue) color space. The color images and/or video of the eyes captured under visible illumination can be used for screening for eye diseases and abnormalities such as cataracts, media opacities in aqueous and vitreous humors, tumors, retinal cancers, and detachment, and the like. Additionally, the color images and/or video can be used with the grayscale images captured under NIR illumination to generate visualizations to assist in the detection of a wide range of eye disease conditions. Additionally, the color images and/or video can be used to screen for autism spectrum disorder, as described in more detail below.

In further examples, the functionalities of the NIR camera 116 and the visible light camera 120 can be combined in an imager equipped with an RGB-Infrared (RGB-IR) filter. In such examples, the RGB-IR filter allows the imager to be used as both a color imager and as a NIR imager depending on a post-processing applied to the images captured by the imager. Such post-processing can be applied based on a selection on the screening device 104.

The screening device 104 can also include one or more display screens, such as a first display screen 122 and a second display screen 124. The one or more display screens can include color LCD (liquid crystal display), active-matrix organic light emitting display (AMOLED), or OLED (organic light-emitting diode) screens. The first and second display screens 122, 124 can be integrated with the screening device 104, or can be external to the screening device 104, and under computer program control of the screening device 104.

The first display screen 122 can include an operator display screen that faces in a direction towards the operator 102, and that is configured to provide information related to the screening tests performed on the screening device 104. In any of the examples described herein, the first display screen 122 facing the operator 102 can be configured to display and/or otherwise provide the output 112 generated by the screening device 104 and/or generated by the screening system 110. The output 112 can include testing parameters, current status, and progress of the screening tests, measurements determined during the tests, images captured or generated during the screening tests, one or more diagnoses determined based on the tests, and/or one or more recommendations. The first display screen 122 can also display information related to the patient such as the patient's medical history, including prior diagnoses and measurements. The first display screen 122 faces in a direction opposite the patient 106, such that the output 112 when displayed on the first display screen 122 is not visible to the patient.

In some examples, the first display screen 122 can be touch sensitive to receive inputs from the operator 102. For example, the first display screen 122 can display a graphical user interface configured to display information to the operator 102 and/or receive input from the operator during a screening test. For example, the first display screen 122 can be used by the operator 102 to enter information regarding the patient, or the screening tests being administered. Further, the first display screen 122 can be configured to display information to the operator 102 regarding the screening test being administered (e.g., parameter settings, progress of screening, options for transmitting data from the screening device 104, one or more measurements, and/or images, videos, or visualizations captured during the screening test.

The second display screen 124 can include a patient display screen that faces in a direction towards the patient 106, and that is configured to display content to the patient 106. The content can include attention-attracting images and/or video to attract attention of the patient and hold the patient's gaze towards the screening device 104. Content corresponding to various vision screening tests can also be presented to the patient 106 on the second display screen 124. For example, the second display screen 124 can display color stimuli to the patient 106 during a color vision screening test, or a Snellen eye chart during a visual acuity screening test. Also, the second display screen 124 can display color stimuli to the patient 106 during a spectrum disorder screening test, as will be described in more detail below.

The screening device 104 can transmit the data captured by the NIR camera 116 and the visible light camera 120, via the network 108, using one or more network interfaces 126. In addition, the screening device 104 can also similarly transmit other testing data associated with the screening tests being administered, (e.g., type of test, duration of test, patient identification and the like). The one or more network interfaces 126 can be operably connected to one or more processing devices 128 of the screening device 104 and can enable wired and/or wireless communications between the screening device 104 and one or more components of the screening system 110, and with one or more other remote systems and/or networked devices.

The one or more network interfaces 126 can include a personal area network component to enable communications over one or more short-range wireless communication channels, and/or a wide area network component to enable communication over a wide area network. In any of the examples described herein, the one or more network interfaces 126 can enable communication between, for example, the one or more processing devices 128 of the screening device 104 and of the screening system 110, via the network 108.

The network 108 can be any type of wireless network or other communication network. Examples of network 108 include the Internet, an intranet, a wide area network (WAN), a local area network (LAN), and a virtual private network (VPN), cellular network connections and connections made using protocols such as 802.11a, b, g, n and/or ac.

The screening system 110 can be configured to receive data from the screening device 104 via the network 108, which is collected during the administration of the screening tests performed by the screening device 104. In some examples, based at least in part on processing the data, the screening system 110 can determine the output 112 associated with the patient 106. For example, the output 112 can include a recommendation and/or diagnosis associated with eye health of the patient 106, based on an analysis of the color image data and/or NIR image data indicative of eye diseases and/or abnormalities of the patient 106. In further examples, the output 112 can include a recommendation and/or diagnosis associated with autism spectrum disorder, based on an analysis of the color image data indicative of a response to visual stimuli emitted by the visible light sources 118 and/or by the second display screen 124. The screening system 110 can communicate the output 112 to the processing devices 128 of the screening device 104 via the network 108. As noted above, in any of the examples described herein one or more such recommendations, diagnoses, or other outputs can be generated, alternatively or additionally, by the screening device 104.

As described herein, the one or more processing devices 128 can include a single processing unit or a number of processing units and can include single or multiple computing units or processing cores. The one or more processing devices 128 can be implemented as one or more microprocessors, microcomputers, microcontrollers, digital signal processors, central processing units, state machines, logic circuitries, and/or any devices that manipulate signals based on operational instructions. The one or more processing devices 128 can include hardware processors and/or logic circuits of any suitable type specifically programmed or configured to execute the algorithms and processes described herein. As shown schematically in FIG. 1, the screening device 104 can also include computer-readable media 130 operably connected to the one or more processing devices 128. The one or more processing devices 128 can be configured to fetch and execute computer-readable instructions stored in the computer-readable media 130, which can program the processing devices 128 to perform the functions described herein.

The computer-readable media 130 can include volatile and nonvolatile memory and/or removable and non-removable media implemented in any type of technology for storage of information, such as computer-readable instructions, data structures, program modules, or other data. Such computer-readable media 130 can include, but is not limited to, RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. The computer-readable media 130 can be a type of computer-readable storage media and/or can be a tangible non-transitory media to the extent that when mentioned, non-transitory computer-readable media exclude media such as energy, carrier signals, electromagnetic waves, and signals per se.

The computer-readable media 130 can be used to store any number of functional components that are executable by the one or more processing devices 128. The functional components can include instructions or programs that are executable by the one or more processing devices 128. When executed, the instructions or programs configure the one or more processing devices 128 to perform actions associated with one or more of the screening tests including screening tests for detecting autism spectrum disorder. For example, the computer-readable media 130 can store one or more functional components for administering the screening tests, such as a patient screening component 132, an image capture control component 134, a data analysis and visualization component 136, and/or an output generation component 138.

In some examples, the patient screening component 132 can be configured to store and/or access the patient data 140 associated with the patient 106. The patient data 140 can include demographic information such as name, age, ethnicity, and the like. When the screening device 104 and/or screening system 110 initiates a screening test, the patient 106 can provide, or the operator 102 can request, from the patient 106 or a guardian of the patient 106 the patient data 140 regarding the patient's demographic information, medical information, preferences, and the like. In such examples, the operator 102 can request the data while the screening is in progress, or before the screening has begun. In some examples, the operator 102 can be provided with predetermined categories associated with the patient 106, such as predetermined age ranges (e.g., newborn to six months, six to twelve months, one to five years old, etc.), and can request the patient data 140 in order to select the appropriate category associated with the patient 106. In other examples, the operator 102 can be provided a free form input associated with the patient data 140. In still further examples, an input element can be provided to the patient 106 directly.

Alternatively, or in addition, the screening device 104 can determine and/or detect the patient data 140 during a screening test. For example, the screening device 104 can include one or more digital cameras or other image capture devices configured to collect images and/or video data of the patient 106, and one or more processors of the screening device 104 can analyze the data to determine the patient data 140, such as the age category of the patient 106.

Additionally, the screening device 104 can determine a distance of the patient 106 from the screening device 104. For example, the screening device 104 can include a range finder, such as an ultra-sonic range finder, an infrared range finder, and/or any other proximity sensor that can be able to determine the distance of the patient 106 from the screening device.

Alternatively, or in addition, the screening device 104 can be configured to transmit the images and/or video data to the screening system 110, via the network 108, for analysis to determine the patient data 140. Further, the patient screening component 132 can be configured to receive, access, and/or store the patient data 140 associated with the patient 106. For example, the patient screening component 132 can store previous patient information associated with the patient 106. For instance, the patient screening component 132 can store previous screening history of the patient 106, including data from previous screening such as color images, NIR images, and/or video of the eyes of the patient 106.

The patient screening component 132 can also receive the patient data 140 and/or can access such information via the network 108. For example, the patient screening component 132 can access an external database, such as screening database 144, storing data associated with the patient 106 and/or other patients. The screening database 144 can be configured to store the patient data 140 in association with a patient ID. When the operator 102 and/or the patient 106 enters the patient ID, the patient screening component 132 can access or receive the patient data 140 stored in association with the patient ID of the patient 106.

In some examples, the patient screening component 132 can be configured to determine which screening tests to administer to the patient 106 based at least in part on the patient data 140. For example, the patient screening component 132 can utilize the patient data 140 to determine a testing category that the patient 106 belongs to (e.g., a testing category based on age, medical history, and the like). The patient screening component 132 can determine the screening tests to administer based on the testing category. For example, when the patient data 140 indicates that the patient 106 is 18 to 24 months old, the patient screening component 132 can determine that the screening tests should include a screening test for autism spectrum disorder as part of a routine checkup. As another example, when the patient data 140 indicates that the patient 106 is a newborn, the selected vision screening tests can include screening for congenital conditions of the eye such as congenital cataracts, retinoblastoma, opacities of the cornea, strabismus and the like. In addition, eye abnormalities can be associated with systemic inherited diseases such as Marfan syndrome and Tay-Sachs disease. For example, a screening test for a characteristic red spot in the eye can indicate Tay-Sachs disease. As another example, when the patient data 140 indicates that the patient 106 is above fifty years old, the patient screening component 132 can determine that the screening tests include screening for onset of cataracts, macular degeneration, and other age-related eye diseases.

The patient screening component 132 can also determine which screening tests to administer based on the patient's medical history. For example, the screening database 144 can store, in the patient data 140, medical history associated with previous screening tests of the patient 106, including test results, images and videos of the patient's eyes, measurements, recommendations, and the like. The patient screening component 132 can access the patient data 140 including medical history from the screening database 144 and determine screening tests to administer to monitor status and changes in previously detected health issues. For example, if the patient 106 was previously diagnosed as having autism spectrum disorder, the patient screening component 132 can determine to administer an autism spectrum disorder test to confirm the prior diagnosis or monitor a progression of the condition. As another example, if a progressive eye disease, such as onset of cataracts or macular degeneration, was detected in a previous screening, further screening can be administered to track the development of the disease. As another example, if the patient 106 had surgery for removal of a tumor of the eyes, the vision screening tests can include screening for further tumors or scarring in the eyes.

The patient screening component 132 can determine a list of screening tests (including both vision and autism spectrum disorder screening tests) to be administered to the patient 106 during a single screening session. The patient screening component 132 can keep track of the screening tests that have already been administered during the screening session, as well remaining screening tests on the list of vision screening tests to be administered.

The computer-readable media 130 can store an image capture control component 134. The image capture control component 134 can be configured to operate the NIR radiation sources 114, the NIR camera 116, the visible light sources 118, and the visible light camera 120 of the screening device 104, so that images of the eyes are captured under specific illumination conditions required for each particular screening tests. As discussed, the NIR radiation sources 114 can include NIR LEDs for illuminating the eyes during capture of grayscale images for measuring the refractive error and/or gaze angle of the eyes of the patient 106, and the visible light sources 118 can include white light LEDs for illuminating the eyes during capture of color images and videos of the eyes by the visible light camera 120.

In examples, the image capture control component 134 can generate commands to operate and control the individual radiation sources, such as the LEDs of the NIR radiation sources 114, as well as the LEDs of the visible light source 118. Control parameters of the LEDs can include intensity, duration, pattern, and cycle time. For example, the commands can selectively activate and deactivate the individual LEDs of the NIR radiation sources 114 and the visible light sources 118 to produce illumination from different angles as needed by the screening tests indicated by the patient screening component 132.

The image capture control component 134 can activate the LEDs of the NIR radiation sources 114 used for measuring the refractive error and/or gaze angle of the eyes of the patient 106 in synchronization with the capture of images of the eyes by the NIR camera 116 during the performance of a vision screening test. Similarly, the image capture control component 134 can activate the LEDs of the visible light sources 118 in synchronization with the capture of color images and video of the eyes by the visible light camera 120 during the performance of an autism spectrum disorder screening test.

The individual radiation sources, such as LEDs of the NIR radiation sources 114 and the visible light sources 118 are controlled by the image capture control component 134 based on control parameters stored in the computer-readable media 130. For instance, control parameters can include intensity, duration, pattern, cycle time, and so forth of the LEDs of the NIR radiation sources 114 and/or the LEDs producing white light from the visible light sources 118. For example, the image capture control component 134 can use the control parameters to determine a duration that individual LEDs of the NIR radiation sources 114 and/or the visible light sources 118 emit radiation (e.g., 50 milliseconds, 100 milliseconds, 200 milliseconds, etc.). Additionally, the image capture control component 134 can utilize the control parameters to alter an intensity and display pattern of NIR LEDs of the NIR radiation sources 114 for the determination of refractive error of the eyes based on photorefraction and/or gaze angle of the eyes.

With respect to intensity, the image capture control component 134 can control parameters to direct the LEDs of the visible light sources 118 to emit light at an intensity that is bright enough to capture a color image of the eyes using the visible light camera 120, while also limiting brightness to avoid or reduce pupil constriction or accommodation. The image capture control component 134 can also control the intensity of the visible light sources 118 to gradually increase the intensity at a certain rate while activating the visible light camera 120 to capture images and/or video of the eyes to record response of the pupils of the patient's eyes to the increasing intensity of illumination. In further examples, the image capture control component 134 can control the visible light sources 118 to generate a stimulus while activating the visible light camera 120 to capture images and/or video of the eyes to record response of the pupils of the patient's eyes to the stimulus for screening for autism spectrum disorder.

Additionally, the image capture control component 134 can order the emission of radiation from the NIR radiation sources 114 and the visible light sources 118 so that the NIR LEDs are activated and the images of the eyes under NIR radiation are captured before the activation of the LEDs of the visible light sources 118. In some examples, this ordering can prevent the constriction of the pupils of the eyes in response to white light impinging upon them, and/or can allow for the capture of images of the internal structures of the eyes without the need for dilating the pupils of the patient 106. In some examples, the image capture control component 134 can additionally control the NIR radiation sources 114 and the visible light sources 118 to generate patterns such as circular patterns, alternating light patterns, flashing patterns, patterns of shapes such as circles or rectangles, and the like to attract the attention of the patient 106, and/or control color LEDs of the visible light sources 118 to display color stimuli such as color dot patterns to the patient 106 during a screening test.

The image capture control component 134 can also control the NIR camera 116 and the visible light camera 120 to capture images and/or video of the eyes of the patient 106 during the administration of the screening tests. For example, the NIR camera 116 can capture data indicative of reflected radiation from the eyes of the patient 106 during the activation of one or more of the NIR radiation sources 114. The data can include grayscale image data and/or video data of the eyes. The image capture control component 134 can also synchronize the visible light camera 120 to capture color images and/or video data of the eyes with the activation of the visible light sources 118 causes the eyes to be illuminated by white light radiation.

In some examples, images of the left and the right eye can be captured under different illumination conditions (e.g., from a different individual source), so that the relative angle of illumination with the optical axis of the particular eye is the same for the left and the right eye. In other examples, images of both eyes can be captured simultaneously under the same illumination. As described herein, the image capture control component 134 of the screening device 104 can generate grayscale images of the eyes illuminated under NIR radiation, and color images of the eyes illuminated under white light. Capturing both the grayscale images and the color images can enable the detection of a wider range of diseases and abnormalities.

In some examples, the computer-readable media 130 can also store a data analysis and visualization component 136. The data analysis and visualization component 136 can be configured to analyze the image and/or video data collected, detected, and/or otherwise captured by components of the screening device 104 (e.g., the NIR camera 116, the visible light camera 120, etc.) during screening tests. For example, the data analysis and visualization component 136 can analyze the data to determine location of the pupils of the eyes in the images and identify a portion of the images corresponding to the pupil (e.g., pupil images). The data analysis and visualization component 136 can analyze the pupil images to determine characterizations of appearance of the pupils in the pupil images. For example, in the instance of the color images and/or videos captured by the visible light camera 120, the characterizations can include values corresponding pupillary response to light flashes from the visible light sources 118 or the second display screen 124, which can indicate a likelihood of autism spectrum disorder.

The data analysis and visualization component 136 can further compare the left pupil images and the right pupil images to determine differences in pupillary response between the left and right pupils. The data analysis and visualization component 136 can also compare the pupillary response with a standard or average pupillary response for patients having the same age as the patient 106. The data analysis and visualization component 136 can also compare the pupillary response to a pupillary response measured during a previous screening test to determine whether a change has occurred in the pupillary response, which may indicate a progression of the autism spectrum disorder of the patient 106.

For example, the data analysis and visualization component 136 can be configured to receive, access, and/or analyze standard data associated with vision and autism spectrum disorder screening. For example, the data analysis and visualization component 136 can be configured to access or receive data from one or more additional databases (e.g., the screening database 144, a third-party database, etc.) storing testing data, measurements, and/or values indicating various thresholds or ranges within which measured values should lie. Such thresholds or ranges can be associated with patients who have the same age as the patient 106, and who do not have autism spectrum disorder, and can be learned or otherwise determined from standard testing.

The data analysis component and visualization component 136 can utilize the standard data for comparison with the average values and differences determined during the screening tests as described above. For example, the standard data can indicate a threshold or a normal range for a pupillary response to a stimulus generated by the visible light sources 118 or the second display screen 124. When the pupillary response from the patient 106 exceeds the threshold, or is outside the normal range, the data analysis component and visualization component 136 generates an output 112 of a likelihood of autism spectrum disorder.

The thresholds and/or ranges associated with the autism spectrum disorder screening test can be based on the testing category of the patient 106 (e.g., the age group or medical history of the patient 106), where the thresholds and/or ranges can be different for different testing categories. The data analysis and visualization component 136 can store as part of the patient data 140, images and/or video captured or generated during the autism spectrum disorder screening tests, measurements (e.g., including pupillary response) associated with the autism spectrum disorder screening tests, test results, and other data in a database (e.g., in the screening database 144) for comparison of data over time. In some examples, the stored images can include images of the face or partial face (e.g., eyes and part of nose) of the patient 106.

For example, the data analysis and visualization component 136 can access a previous pupillary response of the patient 106 from the one or more additional databases (e.g., the screening database 144, a third-party database, and the like). The data analysis and visualization component 136 can compare a current pupillary response to the previous pupillary response to determine a difference. The difference between the current and previous pupillary responses can indicate a progression of the autism spectrum disorder, an increased likelihood of autism spectrum disorder, or a decreased likelihood of autism spectrum disorder.

Based on the comparison with a threshold and/or range described above, the data analysis and visualization component 136 can generate a normal/abnormal or a pass/refer determination for the pupillary response of the patient 106. For example, if all values and differences measured are less than on equal to corresponding thresholds or fall within the corresponding ranges of the standard data, a “normal” or “pass” determination can be made by the data analysis and visualization component 136, and an “abnormal” or “refer” determination made otherwise to indicate a referral for further screening. Alternatively, or in addition, the data analysis and visualization component 136 can generate a normal/abnormal determination for various eye diseases and/or abnormalities screened for during a single screening session.

In examples, the data analysis and visualization component 136 can utilize one or more machine learning techniques to generate a diagnosis or recommendation with regards to autism spectrum disorder. For example, machine learning (ML) models can be trained with normal pupillary responses for various age groups, and abnormal pupillary responses for various age groups labeled as exhibiting autism spectrum disorder. The trained ML models can then generate the output 112 indicating a diagnosis or recommendation regarding autism spectrum disorder when provided an input such as, for example, a pupillary response and/or a video of the patient's pupil during stimulation by the visible light sources 118.

In some examples, a plurality of trained ML models can be used, each ML model being trained to detect a different aspect of autism spectrum disorder such as Asperger's syndrome, Rett syndrome, childhood disintegrative disorder, Kanner's syndrome, and pervasive developmental disorder. In such examples, each ML model outputs a binary present/absent indication to indicate whether the input (pupillary response and/or video of the patient's pupil during stimulation by the visible light sources 118) exhibits the type of autism that the ML model is trained to detect. The data analysis and visualization component 136 can provide a video recording of the eyes as input to each ML model of the plurality of trained ML models for detecting one or more types of autism. In examples, the ML models can be neural networks, including convolutional neural networks (CNNs). In other examples, the ML models can also include regression algorithms, decision tree algorithms, Bayesian classification algorithms, clustering algorithms, support vector machines (SVMs) and the like.

The data analysis and visualization component 136 can also generate visualizations of the eyes using the images and/or video data captured by the visible light camera 120. For example, a visualization can include a sequence of still images, or an animated video comprising the sequence of still images. In some instances, the sequence of images can be captured by the visible light camera 120 while the eyes are illuminated by the visible light sources 118 or the second display screen 124 at a progression of different angles along different axes with respect to the optical axis, including an angle of approximately zero degrees relative to the optical axis. For instance, in some examples, the radiation emitted by the visible light sources 118 or the second display screen 124 can be emitted substantially parallel to and/or substantially along the optical axis. In such examples, the emitted radiation can be coaxial or near coaxial with the optical axis. In some examples, the visualization can include graphics and/or color-coding indicative of areas of the images and/or video data that are flagged as being abnormal.

The computer-readable media 130 can additionally store an output generation component 138. The output generation component 138 can be configured to receive, access, and/or analyze data from the data analysis and visualization component 136 and generate the output 112. For example, the output generation component 138 can utilize the normal/abnormal determinations of the data analysis and visualization component 136 to generate a recommendation in the output 112. The recommendation can indicate whether the screening results of the patient 106 indicate no detection of autism spectrum disorder, or further screening is needed based on an “abnormal” screening result. In addition, the output generation component 138 can incorporate all or a subset of the visualizations generated by the data analysis and visualization component 136 into the output 112 to aid diagnosis of autism spectrum disorder.

Portions of the images and/or video captured by the NIR camera 116 or the visible light camera 120 can also be included in the output 112. Additionally, when abnormality is determined, the output generation component 138 can incorporate a likely diagnosis into the output 112 based on an analysis by the data analysis and visualization component 136. The output 112 can be presented to the operator 102 of the screening device 104 via an interface of the device, such the first display screen 122. As described above, because the first display screen 122 faces in a direction opposite the patient 106, the output 112 is not visible to the patient.

The output generation component 138 can also store the output 112, which can include a recommendation, diagnosis, measurements, captured images/video and/or the generated visualizations in a database, such as the screening database 144, for evaluation by a clinician, or for access during subsequent screenings of the patient 106. The screening database 144 can provide access to authorized medical professionals to enable printing of reports or further assessment of the data related to the screening of the patient 106.

Although FIG. 1 illustrates example processing devices 128 and the computer-readable media 130 storing the patient screening component 132, the image capture control component 134, the data analysis and visualization component 136, the output generation component 138 as components of the screening device 104, in any of the examples described herein, the screening system 110 can include similar components and/or the same components. In such examples, the screening system 110 can include one or more processors 146 and computer-readable memory 148 that are configured to perform the functions of some or all of the components in the computer-readable media 130 of the screening device 104. For example, one or more of the components of the computer-readable media 130 can be included in analysis components 150 of computer-readable memory 148 and be executable by the processors 146.

The screening system 110 can communicate with the screening device 104 using a network interfaces 152, and via the network 108, to receive data from the screening device 104, and to send results (e.g., the output 112) back to the screening device 104. The screening system 110 can be implemented on a computer proximate the screening device 104 or can be remotely located. For example, the screening system 110 can be implemented as a remote cloud server.

The network interfaces 152 can enable wired and/or wireless communications between the components and/or devices shown in environment 100 and/or with one or more other remote systems, as well as other networked devices. For instance, at least some of the network interfaces 152 can include a personal area network component to enable communications over one or more short-range wireless communication channels. Furthermore, at least some of the network interfaces 152 can include a wide area network component to enable communication over a wide area network. Such network interfaces 152 can enable, for example, communication between the screening system 110 and the screening device 104 and/or other components of the environment 100, via the network 108. For instance, the network interfaces 152 can be configured to connect to external databases (e.g., the screening database 144) to receive, access, and/or send screening data using wireless connections.

Wireless connections can include cellular network connections and connections made using protocols such as 802.11a, b, g, and/or ac. In other examples, a wireless connection can be accomplished directly between the screening device 104, the screening system 110, and other external systems and devices using one or more wireless protocols such as Bluetooth, Wi-Fi Direct, radio-frequency identification (RFID), infrared signals, and/or Zigbee. Other configurations are possible. The communication of data to an external database can enable the display and sharing of reports for further assessment of the data acquired from screening the patient 106 by the screening device 104. For example, collected data and corresponding test results can be wirelessly transmitted and stored in a remote database accessible by authorized medical professionals, such as an electronic medical record (EMR) system.

While FIG. 1 depicts the environment 100 as having a single vision screening system, in additional examples, the environment 100 can include any number of local or remote vision screening systems substantially similar to the screening system 110 and configured to operate independently and/or in combination and configured to communicate via the network 108.

As discussed herein, FIG. 1 depicts an example of the screening device 104 that includes components for administering one or more screening tests for eye vision, diseases, and abnormalities as well as for autism spectrum disorder during a single screening session. In some examples, one or more aspects can be implemented on the screening system 110, which is remotely located with respect to the screening device 104. The screening system 110 communicates with the screening device 104 over a network 108. The screening device 104 and its components are described in detail with reference to the remaining figures.

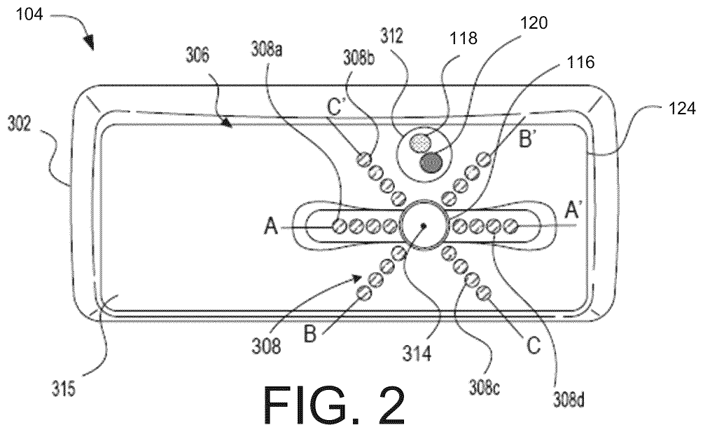

FIG. 2 illustrates an example of the screening device 104 according to some implementations. In this illustrative example, the screening device 104 includes a housing 302 having a first end 306 facing the patient 106. The housing 302 is shaped and sized for handheld and portable use, which improves flexibility of using the screening device 104 on patients as young as newborns. For example, the housing 302 includes a form factor having a compact arrangement and being lightweight. The first end 306 includes the second display screen 124.

The second display screen 124 can cover optical components of the screening device 104, such as an array of LEDs 308 included as part of the NIR radiation sources 114, the NIR camera 116, and an image capture module 312 including the visible light source 118 and the visible light camera 120. Although the visible light source 118 is shown as being proximate the visible light camera 120, in some examples, the visible light source 118 and/or additional white light sources can be disposed at other locations on the housing 302. For example, the visible light source 118 and/or additional white light sources can be disposed at one or more corners 315 of the housing 302, along or proximate one or more sides or edges of the housing 302, and/or at any other location). Since the second display screen 124 is transparent, radiation from the array of LEDs 308 and/or white light from the visible light source 118 of the image capture module 312 can reach the patient's eyes and reflected radiation from the patient's eyes can be received by the NIR camera 116 and/or the visible light camera 120 of the image capture module 312 by traveling through the second display screen 124 without attenuation and change in direction.

The array of LEDs 308 can be comprised of individual NIR LEDs (e.g., NIR LEDS 308a, 308b, 308c, 308d), distributed in a pattern around the NIR camera 116. The NIR LEDs 308a-308d can be arranged along different axes, such as axis A-A′, an axis B-B′, and an axis C-C′. The NIR camera 116 are located substantially along a central axis 314 of the screening device 104. Though the individual NIR LEDs of the array of LEDs 308 are shown as radiating outwards from the NIR camera 116, other patterns of placement of the NIR LEDs of the array of LEDs 308 with more or fewer individual NIR LEDs are also envisioned. The arrangement of NIR LEDs of the array of LEDs 308 can provide eccentric illumination required for measuring refractive error using photorefraction techniques.

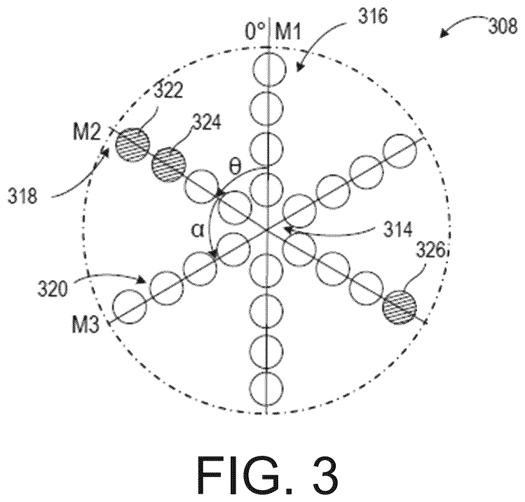

FIG. 3 illustrates an expanded view of the array of LEDs 308. In some examples, the array of LEDs 308 can include more or fewer individual LEDs than those shown in the example. The example arrangement of individual NIR LEDs in the array of LEDs 308 shown in FIG. 3 includes a row 316 of NIR LEDs that extend generally coaxially across the array of LEDs 308 along a first axis M1 of the array of LEDs 308, which can correspond to the axis A-A′ of FIG. 2. The array of LEDs 308 can also include a row 318 along a second axis M2, which can correspond to the axis B-B′ of FIG. 2, at an angle of θ relative to the row 316, and a row 320 along a third axis M3, which can correspond to the axis C-C′ of FIG. 2, at an angle of a relative to the row 318, as shown. The angles θ and a can be any acute angle (e.g., 60 degrees).

In some examples, the axis M1 can also be referred to as a first meridian of the array of LEDs 308, the axis M2 as a second meridian of the array of LEDs 308, and the axis M3 as a third meridian of the array of LEDs 308, and the three axes M1, M2, M3 can intersect at the central axis 314 of the screening device 104. In some examples, additional LEDs can be located along one or more axes M1, M2, M3 spaced away from the array of LEDs 308.

The individual LEDs of the array of LEDs 308 can be activated in a sequence by the image capture control component 134 to generate illumination at different angles or eccentricities. For example, LED 322 can be activated first, followed by the LED 324, adjacent to the LED 322, and so on to LED 326 in a sequence progressively along the axis M2. The activation sequence can move through the individual LEDs along the first axis M1, followed by the second axis M2, and the third axis M3. Also, the LEDs 322 and 324 can also be activated substantially simultaneously in order to simulate a source location of the combined radiation using a diffuser. In such examples, an amount of current applied to the LED 322 and the LED 324 can be further controlled by the image capture control component 134 to achieve a desired simulated source location of the combined radiation, allowing for generation of illumination at additional angles or eccentricities without having to mechanically move the array of LEDs 308.

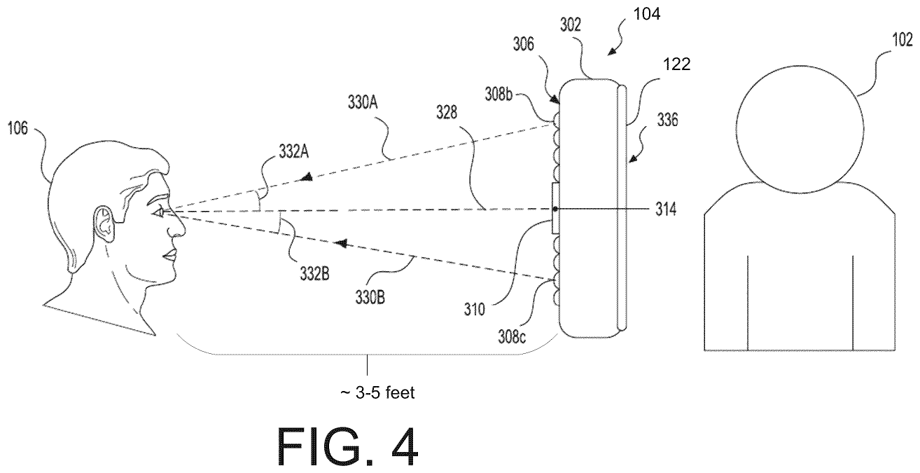

FIG. 4 illustrates an example of the screening device 104 being used by the operator 102 to administer screening tests on the patient 106. The screening device 104 is held by the operator 102 at a distance of about 3 feet to about 5 feet from the patient 106. This can make the various screening tests performed on the screening device 104 more comfortable for the patient, especially when the patient is a child who can be restless and unwilling to remain still.

In some examples, the central axis 314 of the screening device 104 can be substantially aligned or colinear with an optical axis 328 of the patient's eyes, as shown. As discussed above, the image capture control component 134 of the screening device 104 can control the individual radiation sources, such as NIR LEDs 308a-308d or 322, 324 of the array of LEDs 308, to emit radiation. The radiation emitted by the individual LEDs can impinge on the eyes of the patient 106 at different angles relative to the optical axis 328. For example, radiation beam 330A emitted by the NIR LED 308b can subtend an angle 332A with the optical axis 328, while radiation beam 330B emitted by the NIR LED 308c can subtend an angle 332B, different from angle 332A. Activation of the array of LEDs 308, individually or in groups, can be used to generate radiation impinging on the eyes of the patient 106 at different angles. In some examples, the angles 332A, 332B can be approximately zero degrees relative to, for example, the optical axis 328 (e.g., the illumination can be coaxial or near coaxial with the central axis 314).

For each angle of illumination, reflected radiation from the eyes of the patient 106 traveling along the optical axis 328 can be captured by the NIR camera 116 located along the central axis 314 which is aligned with the optical axis 328, to generate images of the eyes under illumination from each angle relative to the optical axis 328. Illumination from different angles with respect to the optical axis 328 can also be generated by the visible light sources 118 using an array or series of individual white light LEDs arranged in a two-dimensional array or a linear pattern, similar to the array of LEDs 308 described above.

The screening device 104 can also include the first display screen 122 disposed on the housing 302 of the screening device 104 on a side 336 opposite the first end 306. The first display screen 122 faces the operator 102 to provide information related to the screening tests to the operator 102. In the illustrative example shown in FIG. 4, the first display screen 122 faces in a direction opposite the patient 106, such that the information displayed on the first display screen 122 is not visible to the patient. In some alternative examples, the first display screen 122 can be separate from (e.g., not attached to the housing 302) the screening device 104, but operably coupled with and under control of the screening device 104.

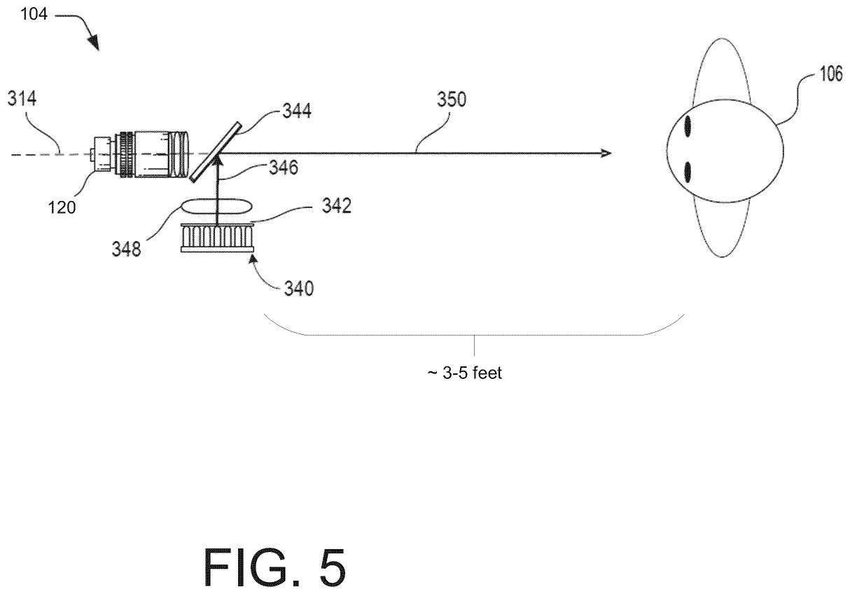

FIG. 5 illustrates an example of components of the screening device 104 being used for an autism spectrum disorder screening test on the patient 106. The components include the visible light camera 120, an array 340 that includes the visible light sources 118, a diffuser 342, and a partial reflector 344. The screening device 104 is held by the operator 102 at a distance of about 3 feet to about 5 feet from the left and right eyes of the patient 106. The autism spectrum disorder screening test performed on the screening device 104 is touchless such that the patient 106 is not required to touch or to be near the screening device. Also, the autism spectrum disorder screening test performed on the screening device 104 does not require any patient involvement such as requiring the patient to perform one or more actions during the test. This can make the various screening tests performed on the screening device 104 more comfortable for the patient, especially when the patient is a child who is restless.

Also, the screening device 104 can perform binocular testing for autism screening by measuring pupillary response to one or more light stimuli in both left and right eyes of the patient 106. This can help improve the sensitivity and specificity of the outputs 112 generated by the screening device 104, in relation to detecting a likelihood of autism spectrum disorder.

The array 340 can be used to generate a light stimulus such as a flash of white light causing a pupillary response in the patient 106 that can be measured, and thereafter compared to one or more metrics for detecting whether the patient 106 exhibits a likelihood of autism spectrum disorder. For example, the data analysis and visualization component 136 can measure a time between a flash from the array 340 and pupil constriction. This data can be collected for both eyes and compared to typical values for subjects having the same age as the patient.

In alternative examples, the second display screen 124 can be used to generate a light stimulus such as an image of a white spot on a black field, or a black dot on a white filed. Autism screening may be provided by recording pupillary responses to the image.

In further examples, the image provided on the second display screen 124 can be used to maintain the patient's attention during the various screening tests that can be performed on the screening device 104, such as when the array 340 is being used to generate the light stimulus. This can be especially helpful when the patient is a child who is restless.

The autism spectrum disorder screening can be performed on the screening device 104 by recording pupillary response to one or more flashes of light or images emitted when screening for eye vision, diseases, and/or abnormalities. Alternatively, the one or more flashes of light or images are generated by the screening device 104 specifically for autism spectrum disorder screening independent of any screening for eye vision, diseases, and/or abnormalities.

As shown in the illustrative example shown in FIG. 5, radiation 346 (e.g., light) emitted by one or more LEDs of the array 340 passes through the diffuser 342 and impinges on the partial reflector 344. The partial reflector 344 can include a beam splitter, an arrangement of mirrors, a prism, or any other optical component configured to reflect a first portion of the radiation impinging on it while transmitting a second portion of the radiation. In some examples, the partial reflector 344 is placed at an angle of approximately 45 degrees with respect to the central axis 314 of the screening system 400 which can be substantially aligned or coaxial with an optical axis of the eyes of a patient 106, as discussed with reference to FIG. 4.

As shown in FIG. 5, the central axis 314 is also substantially aligned or coaxial with a lens of the visible light camera 120. The diffuser 342 can include a blur smoothing filter for the radiation 346 emitted by the LEDs in the array 340. In some examples, a lens 348 can be configured to focus the radiation 346 emitted by the array 340 to impinge on the partial reflector 344. Additional optical components can be included to modify the radiation 346 reaching the partial reflector 344. At least a portion 350 of the radiation 346 reflects off of the partial reflector 344 and is directed to one or both eyes of the patient 106. While the portion 350 of the radiation 346 is directed at the eyes of the patient 106, the visible light camera 120 can capture one or more images and/or video of the eyes of the patient 106. In examples, the images and/or video can depict radiation that is reflected by the pupils of the eyes of the patient 106.

As described herein with reference to FIGS. 2-5, the screening device 104 can include NIR radiation sources and sensors to capture NIR images and video of the eyes of the patient 106, as well as white light sources and a color camera to capture color images and video of the eyes of the patient 106. The screening device 104 can also capture images of the eyes while under illumination from radiation sources at different angles with respect to the optical axis of the eyes. While the radiation sources have been described as comprising infrared or near-infrared (NIR) radiation sources, in additional examples, the radiation sources can include LEDs emitting radiation at different wavelengths, and the radiation sensors can capture images of the eyes while illuminated by radiation at different wavelengths and/or different wavelength bands (e.g., infrared, NIR, visible light, and the like). The different wavelengths of radiation in the visible spectrum can include wavelengths corresponding to specific colors. In such examples, the screening device 104 can detect diseases and/or abnormalities of the eyes that are more apparent in images captured under illumination of specific wavelengths. In addition, since colored light and white light are also forms of electromagnetic radiation, the term “radiation source” as used herein, can refer to both visible light emitters as well radiation emitters in the infrared/NIR ranges of the electromagnetic spectrum.

A pupillary response, also known as pupillary light reflex/response (PLR), is a reflex that controls the diameter of the pupil, in response to the intensity (luminance) of light that falls on the retina in the back of the eye, thereby assisting in adaptation of vision to various levels of lightness and darkness. A greater intensity of light causes the pupil to constrict (also called miosis or myosis), thereby allowing less light into the eye. A lower intensity of light causes the pupil to dilate (also called mydriasis or expansion), thereby allowing more light into the eye. Thus, the pupillary light reflex regulates the intensity of light entering the eye.

Pupillary response can be used to assess brain stem function. Abnormal pupillary response can be caused by optic nerve injury, oculomotor nerve damage, brain tumors, medications such as barbiturates, traumatic brain injury. More recently, studies have found that abnormal pupillary response can be symptomatic of neurodevelopmental disorders such as autism spectrum disorder, attention deficit disorder, hyperactivity, and down syndrome.

Additionally, it has been found that abnormal pupillary response for patients who have autism spectrum disorder changes with age. For example, it has been found that children diagnosed with autism spectrum disorder are hypertensive to light stimulus. In contrast, adults diagnosed with autism spectrum disorder are hyposensitive to light stimulus.

Abnormal pupillary response has been found in children as young as 2 to 6 months old who were later diagnosed with autism spectrum disorder. The abnormal pupillary response can include abnormalities such as constriction time (e.g., children who have autism spectrum disorder exhibit shorter constriction times in response to a light stimulus); amplitude of constriction (e.g., children who have autism spectrum disorder exhibit a larger degree or amount of pupil constriction in response to a light stimulus); and time for returning to a baseline pupil diameter (e.g., children who have autism spectrum disorder exhibit faster pupil dilation).

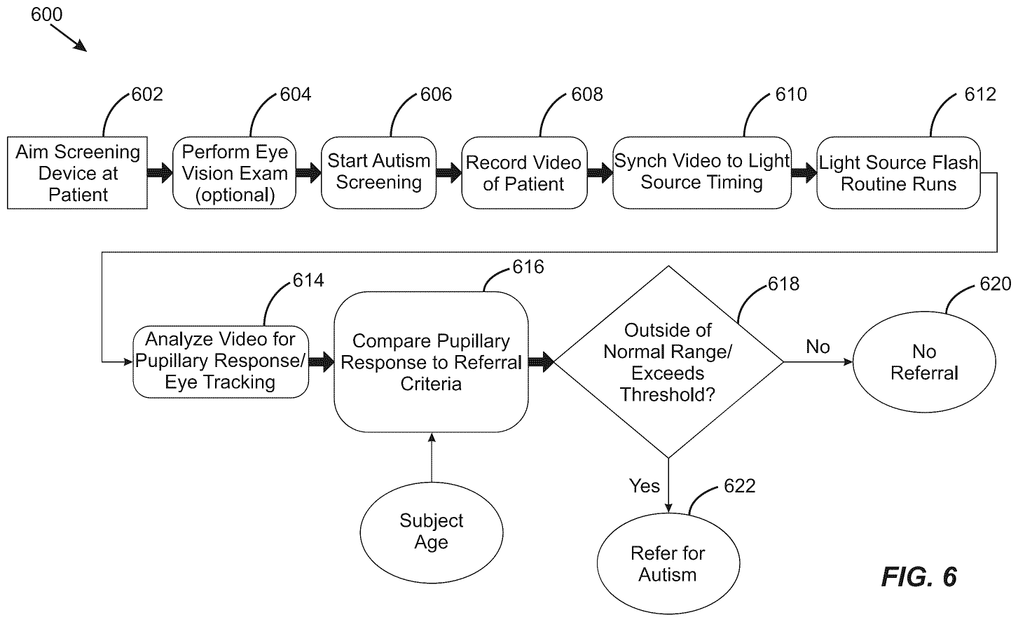

FIG. 6 schematically illustrates an example of a method 600 of screening for autism spectrum disorder than can be performed on the screening device 104. The method 600 can be performed on the screening device 104 to screen for autism spectrum disorder at an earlier age than typical autism screening tests to enable earlier intervention. Also, the method 600 can be performed on the screening device 104 to remove subjectivity from the diagnosis.

The method 600 includes an operation 602 of aiming the screening device 104 at the patient 106. The patient 106 is a child who can have an age ranging from about two months to about four years old. In some examples, the method 600 can be performed on the screening device 104 for patients who are older than four years old. The screening device 104 can be held at a distance of about 3 feet to about 5 feet from the patient 106 (see FIG. 5). Accordingly, the method 600 is touchless such that the patient 106 is not required to touch or to be in close proximity to the screening device 104. Also, the method 600 does not require any patient involvement such as requiring the patient to perform one or more actions. This can make the method 600 more comfortable for the patient, especially when the patient is a child.

Also, the method 600 includes binocular testing for autism screening by measuring pupillary response to one or more light stimuli in both left and right eyes of the patient 106. This can help improve the sensitivity and specificity of the outputs 112 generated by the screening device 104 relating to detection of autism spectrum disorder, as further described below.

As discussed above, the screening device 104 includes a housing 302 that is portable and handheld. This enables the operator 102 to follow the patient 106, which makes it easier to perform the autism spectrum disorder screening test, especially when the patient 106 is a young child. In some instances, the operator 102 may sit on the floor with a child who is not cooperating and use the screening device 104 to screen for autism spectrum disorder. In contrast, devices such as wall mounted devices or stationary devices, do not provide such flexibility in allowing the operator to conduct a screening test.

In some examples, the method 600 can include of an operation 604 of performing a screening test for eye vision, diseases, and/or abnormalities. As shown in FIG. 6, operation 604 is optional. In examples where the method 600 includes operation 604, the autism spectrum disorder screening test is performed as part of standard vision screening for young children. Alternatively, the autism spectrum disorder screening can be performed on the screening device 104 as a separate evaluation protocol independent of eye vision screening.

For example, operation 604 can include performing one or more vision screening tests such as visual acuity tests, refractive error tests, eye accommodation tests, dynamic eye tracking tests, color vision screening tests, and/or any other vision screening tests. In further examples, operation 604 can include performing one or more screening tests for eye diseases and abnormalities such as glaucoma, cataracts, macular degeneration, media opacities in aqueous and vitreous humors, tumors, retinal cancers, and detachment, and the like.

As further shown in FIG. 6, the method 600 includes an operation 606 of starting the autism spectrum disorder screening test. In some examples, operation 606 includes starting the autism spectrum disorder screening test following a selection of a screening test on the first display screen 122 by the operator 102. As described above, when the patient data 140 (see FIG. 1) associated with the patient 106 indicates that the patient 106 is a child, the patient screening component 132 can recommend performing the autism spectrum disorder screening test as part of a routine checkup. In some examples, selectable options are displayed on the first display screen 122 allowing the operator 102 to accept or decline the recommendation to perform the autism spectrum disorder screening test. In such examples, operation 606 can include starting the autism spectrum disorder screening test upon acceptance of the recommendation.

Alternatively, operation 606 can include automatically starting the autism spectrum disorder screening test. For example, operation 606 can include automatically starting the autism spectrum disorder screening test when the patient screening component 132 recommends the screening test as part of a routine checkup based on the patient data 140.

As further shown in FIG. 6, the method 600 includes an operation 608 of capturing a video of the pupils of the patient 106 by using the visible light camera 120, an operation 610 of synchronizing the video to a light stimulus, and an operation 610 of emitting the light stimulus using the visible light sources 118 and/or the second display screen 124. In some examples, the operations 608-612 are performed at substantially the same time such that they are simultaneous. Alternatively, the operations 608-612 can be performed sequentially in the order shown.

The method 600 includes an operation 614 of analyzing the captured video for measuring pupillary response to the light stimulus. Operation 614 can include measuring a time for pupil constriction, measuring an amplitude of the pupil constriction, and/or measuring a time for returning to a baseline pupil diameter after the light stimulus.

In some examples, operation 614 can include eye tracking while an image is displayed on the second display screen 124. For example, studies have shown that autism patients typically focus on different portions of the human face than non-autistic patients such as the eyes versus the nose, mouth, and the like. Accordingly, the screening device 104 can use either the NIR camera 116 or the visible light camera 120 to perform eye tracking in combination with presenting images of human faces on the second display screen 124. In some further examples, the images of human faces can be of one or more family members of the patient 106 (e.g., a parent or guardian of the patient 106). In some examples, the screening device 104 performs eye tracking in response to presenting images of human faces to confirm or validate a recommendation or diagnosis of autism spectrum disorder based on pupillary response.

In some examples, operation 614 can include measuring eye saccades. Eye saccades are quick, simultaneous movements of both eyes between two or more phases of fixation in the same direction. As an illustrative example, operation 614 can include performing eye tracking to measure the number of eye saccades detected from the eyes of the patient 106 during a given time period. In some examples, operation 614 can include evaluating a latency of eye tracking and saccades to correct for slow or inaccurate tracking of moving objects. The eye saccades measurement(s) can be used to confirm or validate a recommendation or diagnosis of autism spectrum disorder based on pupillary response.

Still referring to FIG. 6, the method 600 includes an operation 616 of comparing the pupillary response measured in operation 614 to one or more referral criteria. As shown in FIG. 6, the one or more referral criteria are based on at least the age of the patient because, as described above, pupillary response to light stimuli by autistic patients changes with age. For example, children having autism spectrum disorder are hypertensive to light stimulus, while adults having autism spectrum disorder are hyposensitive to light stimulus.

The referral criteria can include one or more normal ranges such as a normal time range for pupil constriction, a normal amplitude range for pupil constriction, and/or a normal time range for returning to a baseline pupil diameter. Operation 616 can include comparing the pupillary response measured in operation 614 to one or more of these normal ranges.

Additionally, or alternatively, the referral criteria can include one or more thresholds such as a time threshold for pupil constriction, an amplitude threshold for pupil constriction, and/or a time threshold for returning to a baseline pupil diameter. Operation 616 can include comparing the pupillary response measured in operation 614 to one or more of these thresholds.

The method 600 further includes an operation 618 of determining whether the pupillary response measured in operation 614 is outside of a normal range defined by the one or more referral criteria, or exceeds a threshold set by the one or more referral criteria.

When the pupillary response measured in operation 614 is within a normal range defined by the one or more referral criteria or does not exceed a threshold set by the one or more referral criteria (i.e., “No” in operation 618), the method 600 proceeds to an operation 620 of issuing an output 112 as a normal or pass result. The output 112 can include a recommendation not to refer the patient 106 for further testing for autism spectrum disorder.

Otherwise, when the pupillary response measured in operation 614 is outside of a normal range defined by the one or more referral criteria, or exceeds a threshold set by the one or more referral criteria (i.e., “Yes” in operation 618), the method 600 can proceed to an operation 622 of issuing an output 112 as an abnormal or fail result. The output 112 can include a recommendation to refer the patient 106 for additional testing for autism spectrum disorder.

The various embodiments described above are provided by way of illustration only and should not be construed to be limiting in any way. Various modifications can be made to the embodiments described above without departing from the true spirit and scope of the disclosure.

What is claimed is:

1. A device for screening for autism spectrum disorder, the device comprising:
a housing configured for portable use;
one or more cameras mounted to the housing;
one or more light sources mounted to the housing;
one or more processing devices housed inside the housing; and
computer-readable media storing software instructions that, when executed by the one or more processing devices, cause the one or more processing devices to:
capture a video of one or more eyes using the one or more cameras;
emit a light stimulus using the one or more light sources;
synchronize the video to the light stimulus;
analyze the video for measuring a pupillary response to the light stimulus;
compare the pupillary response to one or more metrics; and
generate an output based on comparing the pupillary response to the one or more metrics, the output including a recommendation related to autism spectrum disorder.

2. The device of claim 1, wherein the computer-readable media further stores software instructions which, when executed by the one or more processing devices, further cause the one or more processing devices to:
perform a screening test for eye vision, an eye disease, or an eye abnormality.

3. The device of claim 1, wherein the pupillary response is measured for both left and right eyes.

4. The device of claim 1, wherein the one or more light sources include an array of light-emitting diodes that generate the light stimulus as a flash of white light.

5. The device of claim 1, wherein the one or more light sources include a display screen that generates the light stimulus as an image.

6. The device of claim 1, wherein the one or more light sources include near-infrared light emitters for measuring refractive error of the one or more eyes.

7. The device of claim 1, wherein the pupillary response includes at least one of a time measurement of pupil constriction, an amplitude measurement of the pupil constriction, and a time measurement for returning to a baseline pupil diameter.

8. The device of claim 1, wherein the computer-readable media further stores software instructions which, when executed by the one or more processing devices, further cause the one or more processing devices to:
capture the video when the housing is held about 3 feet to about 5 feet away from the one or more eyes.

9. The device of claim 1, wherein the computer-readable media further stores software instructions which, when executed by the one or more processing devices, further cause the one or more processing devices to:
perform eye tracking to validate the output.

10. A method of screening for autism spectrum disorder, the method comprising:
capturing a video of one or more eyes;
emitting a light stimulus;
synchronizing the video to the light stimulus;
analyzing the video for measuring a pupillary response to the light stimulus;
comparing the pupillary response to one or more metrics; and
generating an output based on comparing the pupillary response to the one or more metrics, the output including a recommendation related to autism spectrum disorder.

11. The method of claim 10, further comprising:
performing a screening test for eye vision, an eye disease, or an eye abnormality.

12. The method of claim 10, further comprising:
generating the light stimulus as a flash of light using an array of light-emitting diodes.

13. The method of claim 10, further comprising:
generating the light stimulus as an image on a display screen.

14. The method of claim 10, wherein the pupillary response includes at least one of a time measurement of pupil constriction, an amplitude measurement of the pupil constriction, and a time measurement for returning to a baseline pupil diameter.

15. The method of claim 10, further comprising:
generating the output when the pupillary response is outside a range of values for a normal pupillary response.

16. The method of claim 10, further comprising:
performing eye tracking to validate the output.

17. A non-transitory computer-readable media storing data instructions, which when executed by one or more processing devices, cause the one or more processing devices to:
capture a video of one or more eyes;
emit a light stimulus;
synchronize the video to the light stimulus;
analyze the video for measuring a pupillary response to the light stimulus;
compare the pupillary response to one or more metrics; and
generate an output based on comparing the pupillary response to the one or more metrics, the output including a recommendation related to autism spectrum disorder.

18. The non-transitory computer-readable media of claim 17, further comprising additional data instructions, which when executed by the one or more processing devices, further cause the one or more processing devices to:
perform a screening test for eye vision, an eye disease, or an eye abnormality.

19. The non-transitory computer-readable media of claim 17, wherein the pupillary response includes at least one of a time measurement of pupil constriction, an amplitude measurement of the pupil constriction, and a time measurement for returning to a baseline pupil diameter.

20. The non-transitory computer-readable media of claim 17, further comprising additional data instructions, which when executed by the one or more processing devices, further cause the one or more processing devices to:
perform eye tracking to validate the output.
